(19) United States
(12) Patent Application Publication
(10) Pub. No.: US 2008/0033162 A1
(43) Pub. Date: Feb. 7, 2008

(54) Water-Soluble Cellulose Derivative Comprising A Ligand

(75) Inventors: Leif Olof Karlson, Stenungsund (SE); Behrouz Syrous, Stenungsund (SE)

Correspondence Address: AKZO NOBEL INC., INTELLECTUAL PROPERTY DEPARTMENT, 120 WHITE PLAINS ROAD 3RD FLOOR, TARRTOWN, NY, 10591, US

(73) Assignee: Akzo Nobel N.V., Amhem (NL)

(21) Appl. No.: 11/630,807

(22) PCT Filed: Jun. 27, 2005
(86) PCT No.: PCT/EP05/52982

§ 371 Date: Dec. 27, 2006

Related U.S. Application Data
(60) Provisional application No. 60/588,788, filed on Jul. 19, 2004.

(30) Foreign Application Priority Data
Jun. 28, 2004 (EP) 04076866.5

(51) Int. Cl. C08B 11/00; C07H 3/00; C09D 101/08; C09D 101/10; C09D 101/26; C09D 101/28

(52) U.S. Cl. 536/56; 106/162.71; 536/124

(57) ABSTRACT

The invention relates to novel cellulose derivatives comprising a ligand capable of forming a complex with a polyvalent metal ion. The invention further pertains to a process of preparing such cellulose derivatives.

The invention relates to water-soluble cellulose derivatives.

Water-soluble cellulose derivatives are known in the art. Typical examples of cellulose derivatives are cationic, non-ionic, and anionic cellulose derivatives. These cellulose derivatives may generally be used as viscosity agents, e.g. in coating compositions. In general, it is desirable to incorporate small amounts of the cellulose derivative into the coating composition and at the same time obtain a coating composition with desirable rheological properties. Use of small amounts of a high-molecular weight cellulose derivative leads to a coating composition having a high viscosity. However, these high-molecular weight cellulose derivatives cause the coating composition to have poor application properties, resulting in the presence of brush marks, poor hiding, and a high tendency for spattering when the coating composition is applied onto a substrate. Coating compositions comprising low-molecular weight cellulose derivatives exhibit better application properties, but need to be added in relatively large amounts in order to obtain the desired viscosity, which is economically unattractive. In order to improve its rheological properties, low-molecular weight cellulose derivatives can be modified by the incorporation of hydrophobic groups. However, coating compositions generally comprise surfactants or colorants which can interact with the hydrophobically-modified cellulose derivative, causing an undesirable reduction in the viscosity of the coating composition.

It is therefore an object of the present invention to provide a novel cellulose derivative. It is a further object to provide a coating composition comprising the novel cellulose derivative which has improved rheological properties.

This object is achieved by providing a water-soluble cellulose derivative comprising a ligand having at least two carboxylic acid groups or a ligand having at least one 5- or 6-membered ring and having at least two nitrogen atoms. In one embodiment of the invention, the ligand has two carboxylic acid groups and is either of the formulae (I) and (II): 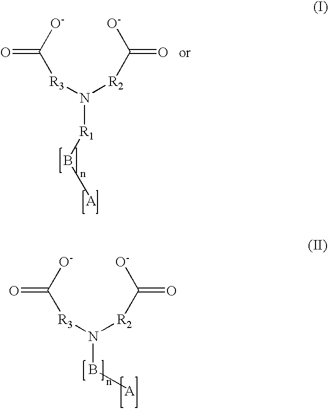
wherein R₁ is a hydrocarbon having 1 to 8 carbon atoms, optionally comprising at least one hydroxyl group, R₂ and R₃ may be the same or different and are selected from a hydrocarbon having 1 to 8 carbon atoms, [B] is attached to the cellulose backbone of the cellulose derivative [A] and selected from O, OC(O), C(O)O, C(O)—NH, NHC(O), NH—C(O)—CH₂—O, O—C(O)—NH—R₄—NH—C(O)—O, wherein R₄ is selected from substituted or unsubstituted C₁-C₈ alkylene and C₃-C₄ cycloalkyl, S, OSO₃, OPO₃, NH, or NR₅, wherein R₅ is a C₂-C₆ acyl or a C₁-C₄ alkyl radical, and wherein n is 0 or 1. Preferably, B is O. It is further preferred that R₂ and R₃ are independently selected from the group consisting of methylene, ethylene, and propylene. R₁ preferably is a hydrocarbon having 1 to 3 carbon atoms or a hydrocarbon having 1 to 3 carbon atoms and at least one hydroxyl group, in particular R₁ preferably is methylene, ethylene, propylene, hydroxymethylene, 1-hydroxyethylene, 2-hydroxyethylene, 1-hydroxypropylene, 2-hydroxypropylene, 3-hydroxypropylene, and most preferably R₁ is 2-hydroxypropylene.

In another embodiment of the invention the ligand has at least one 5- or 6-membered ring and at least two nitrogen atoms. Preferably, the ring structure comprises at least one singly unsaturated C═C bond and more preferably forms a conjugated system. In a preferred embodiment, the ligand is connected to the cellulose derivative via a carbon atom forming part of the ring, and not to any one of the nitrogen atoms. In this way, the complex formed with metal ions (vide supra) generally is more stable.

Typical examples of such a ligand are ligands selected from the group consisting of imidazole, pyrimidine, purine, phenantroline, bipyridine, terpyridine, and derivatives thereof. Preferably, the ligand is selected from the group consisting of imidazole, terpyridine, and derivatives thereof. The term “derivatives” refers to groups attached to the ring structure of any of the mentioned ligands, such as for example hydrocarbons having between 1 and 10 carbon atoms, which optionally comprise a functional group such as hydroxyl or amine.

The water-soluble cellulose derivative according to the invention is capable of forming a complex with a polyvalent metal ion. Preferably, the polyvalent metal ion is selected from the group consisting of the transition metals, Mg²⁺, Ca²⁺, and Al³⁺. The transition metals preferably are Zr⁴⁺, Co²⁺, Fe²⁺, Fe³⁺, Mn²⁺, Mn⁴⁺, Zn²⁺, Cu²⁺, and Cr³⁺. The most preferred polyvalent metal ions are Mg²⁺, Ca²⁺, Zr⁴⁺, Fe²⁺, Fe³⁺, Cu²⁺ and Zn²⁺. In this way, complexation of two cellulose derivatives comprising a ligand according to the invention with one polyvalent metal ion links the two cellulose derivatives to each other. Such linkage may change the rheological properties of the cellulose derivative, e.g. the viscosity of a solution of this cellulose derivative is increased. A further advantage is that these novel cellulose derivatives are not negatively influenced by the presence of a surfactant or colorant which interacts with conventional cellulose derivatives. This renders the cellulose derivatives of the present invention highly suitable for use as viscosity agents in coating compositions comprising the said surfactants and/or colorants.

In the context of the present application the language “water-soluble cellulose derivative” means that the cellulose derivative of the invention is at least partially soluble in water. Generally, the solubility of the cellulose derivative in water is at least 0.05 g/l. It is noted that cross-linked cellulose derivatives are less preferred, as they are generally not water-soluble and/or have undesirable rheological properties, in particular for use in coating compositions.

The cellulose derivative according to the invention generally has an average molecular substitution (also referred to as MS) of ligands of at least 0.001, preferably at least 0.005, and most preferably at least 0.02, and of at most 1.0, preferably at most 0.5, and most preferably at most 0.35.

The cellulose derivative may have only ligands substituted onto the cellulose backbone. It may also be desirable to introduce other substituents onto the cellulose backbone or onto other reactive hydroxyl groups of the cellulose derivative. These substituents are generally known to the man skilled in the art, and may be anionic, non-ionic or cationic. The cellulose derivative may comprise at least one anionic, at least one non-ionic and/or at least one cationic substituent.

Examples of non-ionic groups are hydroxyethyl, hydroxypropyl, methyl, and ethyl. If the non-ionic group is hydroxyethyl or hydroxypropyl, the cellulose derivative has an MS of the non-ionic group of at least 0.2, preferably at least 0.3, and most preferably at least 0.4, and of at most 4.5, preferably at most 4.0, and most preferably at most 3.5. In the case the non-ionic group is ethyl or methyl, the cellulose derivative has an average degree of substitution (also referred to as DS) of the non-ionic groups is at least 0.05, preferably at least 0.1, more preferably at least 0.15, and most preferably at least 0.2, and at most 1.5, preferably at most 1.2, more preferably at most 1.0, and most preferably at most 0.8.

Examples of anionic groups are carboxyalkyl, sulphonate (e.g. sulphoethyl), phosphate, and phosphonate groups. Of the anionic groups carboxyalkyl and in particular carboxymethyl are most preferred. Generally, the average DS of carboxymethyl groups is at least 0.05, preferably at least 0.1, more preferably at least 0.15, and most preferably at least 0.2, and at most 1.2, preferably at most 1.0, more preferably at most 0.8, and most preferably at most 0.6.

The cationic group can be a primary, secondary, tertiary amine or quaternary ammonium group. Examples of such cationic groups can be gleaned from U.S. Pat. No. 6,281,172. Generally, the degree of substitution of cationic groups is at least 0.01, preferably at least 0.02, and most preferably at least 0.05, and at most 1.0, preferably at most 0.5, and most preferably at most 0.35

Generally, the molecular weight of the cellulose derivative of the invention is at least 10,000 Dalton, preferably at least 35,000 Dalton, and most preferably at least 50,000 Dalton, and at most 2,000,000 Dalton, preferably at most 1,200,000 Dalton, and most preferably at most 800,000 Dalton.

In the case that the cellulose derivative of the present invention is used in a coating composition, the molecular weight of the cellulose derivative is at least 10,000 Dalton, preferably at least 35,000 Dalton, and most preferably at least 50,000 Dalton, and at most 1,200,000 Dalton, preferably at most 800,000 Dalton, and most preferably at most 500,000 Dalton.

The water-soluble cellulose derivatives comprising a ligand having at least two carboxylic acid groups in accordance with the present invention can be prepared by a process comprising the steps of:
(a) reacting an epoxide according to any one of the formulae: 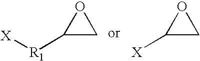
with a secondary amine of the formula: 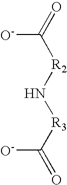
to form a ligand according to one of the formulae: 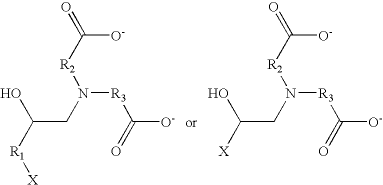
wherein R₁ is a hydrocarbon having 1 to 6 carbon atoms, optionally comprising at least one hydroxyl group, R₂ and R₃ may be the same or different and are selected from a hydrocarbon having 1 to 8 carbon atoms, and X is a halogen selected from Cl⁻, Br⁻, and I; and
(b) subsequently reacting the ligand with a cellulose derivative according to the formula: 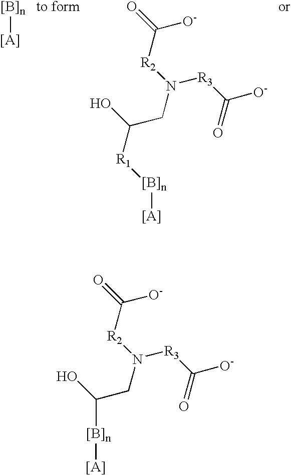
wherein R₁, R₂ and R₄ are as defined above, and [B] is attached to the cellulose backbone of the cellulose derivative [A] and selected from 0, OC(O), C(O)O, C(O)—NH, NHC(O), NH—C(O)—CH₂—O, O—C(O)—NH—R₄—NH—C(O)—O, wherein R₄ is an C₁-C₈ alkylene, S, OSO₃, OPO₃, NH, or NR₅, wherein R₅ is a C₂-C₆ acyl or a C₁-C₄ alkyl radical, and wherein n is 0 or 1.

Step (a) of the process is typically carried out in the presence of a first solvent. The first solvent may be any solvent suitable to dissolve the reactants. Examples of such first solvents are water, methanol, ethanol, 1-propanol, 2-propanol, dimethyl sulphoxide, dimethyl formamide, and mixtures thereof.

Step (b) of the process is typically carried out in the presence of a second solvent. The second solvent may be any solvent suitable to dissolve the cellulose derivative. The second solvent may be the same as the first solvent or a different solvent. It is preferred that the second solvent is chosen such that it will mix intimately with the first solvent, and that no phase separation occurs. Examples of such solvents are water, methanol, ethanol, 1-propanol, 2-propanol, dimethyl sulphoxide, dimethyl formamide, and mixtures thereof.

In a further embodiment of the invention, a water-soluble cellulose derivative comprising a ligand having at least one 5- or 6-membered ring and having at least two nitrogen atoms in accordance with the present invention can be prepared by the process described above, except that the secondary amine is replaced with a precursor of the ligand having at least one 5- or 6-membered ring and having at least two nitrogen atoms as described above. The precursor of the ligand generally refers to compounds that can react with the cellulose derivative to form the ligand. Examples of such precursors are imidazole, pyrimidine, purine, phenantroline, bipyridine, terpyridine and derivatives thereof, and any of these precursors having at least one reactive group, such as chlorine, bromine, or iodine, attached to a carbon located in the ring structure. Examples of precursors without a reactive group are imidazole, 2,4-dimethyl imidazole, 1,7-phenantroline, 4,7-phenantroline, 1,10-phenantroline, pyrimidine, purine, 2,2-dipyridyl, and 2, 2:6,2″-terpyridine.

Examples of precursors having at least one reactive group are 4′-chloro-2,2′:6′,4′-terpyridine, 2,2-pyridylamine, 4-bromo-5-methyl-1H-imidazole, 4-bromo-2-methyl-1H-imidazole, and 4-bromo-1H-imidazole. 4′-chloro-2,2′:6′,4′-terpyridine and 4-bromo-1H-imidazole are preferred.

It is also envisaged to leave out step (a) and add the precursor and/or the precursor having the reactive group in step (b).

The cellulose derivatives of the invention can be used as a viscosity agent or as a sequestering agent. The cellulose derivatives of the present invention are preferably used as viscosity agents in coating compositions. The coating composition can be any coating composition known to the man skilled in the art. These cellulose derivatives, and in particular the water-soluble cellulose derivatives, can be particularly advantageously used in water borne coating compositions. Preferably, the coating composition comprises the non-ionic cellulose derivative according to the invention, and a salt of a polyvalent metal ion. Preferably, the polyvalent metal ion is selected from the group consisting of the transition metals, Mg²⁺, Ca²⁺, and Al³⁺. The transition metals preferably are Zr⁴⁺, Co²⁺, Fe²⁺, Fe³⁺, Mn²⁺, Mn⁴⁺, Zn²⁺, Cu²⁺, and Cr³⁺. The most preferred polyvalent metal ions are Mg²⁺, Ca²⁺, Zr⁴⁺, Fe²⁺, Fe³⁺, Cu²⁺, and Zn²⁺.

The invention is illustrated by the following examples.

### EXPERIMENTAL

### Example 1

Sodium salt of iminodiacetic acid (IDA), 5.88 g (29.3 mmol), was dissolved in 24 g deionized water to which 1.3 g sodium hydroxide (32.5 mmol) was added. The solution was subsequently heated to 37° C. and 2.67 ml (34 mmole) epichlorohydrin (ECH) was added dropwise from a dosimate, so that the temperature remained between 41-46° C. Following the addition of ECH, the temperature was increased to 60° C. after 5 minutes and kept there for 60 minutes before the mixture was cooled to room temperature. Chloride analysis by titration showed a quantitative conversion of ECH to the corresponding ligand (see reaction II).

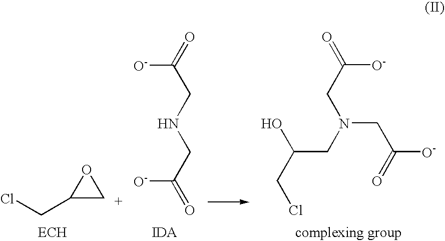

The whole solution comprising the ligand was transferred to a dropping funnel and over a 2-hour period was added to an alkalized 2.5% w/w ethyl hydroxyethyl cellulose (EHEC) solution (0.1 g NaOH and 8.34 g EHEC (MS(EO) is 2.1; DS(ethyl) is 0.8; Mw is 200,000) in 320 g of a mixture of 48% w/w 2-propanol (IPA) in deionized water), which EHEC solution was kept under a nitrogen atmosphere and had a temperature of 60° C.

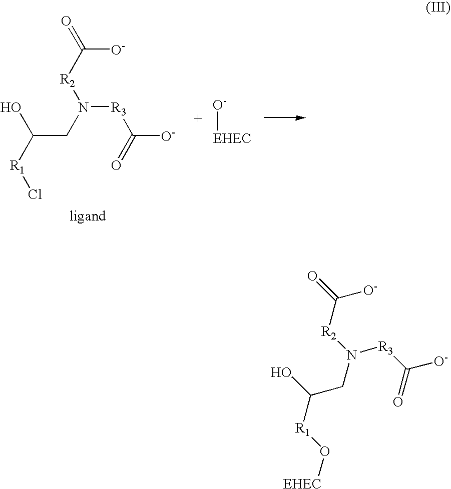

After addition of the ligand-containing solution, the resulting mixture was kept at this temperature for 2 hours, after which it was quenched with acetic acid. To get rid of unreacted components and low-molecular weight by-products, the clear solution was dialyzed against excess water of Millipore quality for seven days with repeated exchange of the dialysis water. The dialysis was performed using a Spectra/Por® membrane tubing with a molecular weight cut-off of 6-8,000. After the dialysis the polymer was recovered by freeze-drying.

The nitrogen content of the new product was determined by Kjelldahl titration to be 0.16% w/w, based on the total weight of the resulting polymer. This corresponds to a MS (ligand) of 0.031.

### Example 2

Sodium salt of iminodiacetic acid (IDA), 5.88 g (29.3 mmol), was dissolved in 24 g deionized water to which 1.3 g sodium hydroxide (32.5 mmol) was added. The solution was subsequently heated to 37° C. and 2.67 ml (34 mmole) epichlorohydrin (ECH) was added dropwise from a dosimate, so that the temperature remained between 41-46° C. Following addition of ECH, the temperature was increased to 60° C. after 5 minutes and kept there for 60 minutes before the mixture was cooled to room temperature. Chloride analysis by titration showed a quantitative conversion of ECH to the corresponding ligand.

The whole solution comprising the ligand was transferred to a dropping funnel and over a 2-hour period was added to an alkalized 2.5% w/w ethyl hydroxyethyl cellulose (EHEC) solution (0.1 g NaOH and 8.34 g EHEC (MS(EO) is 2.1; DS(ethyl) is 0.8; Mw is 200,000) in 320 g deionized water), which EHEC solution was kept under a nitrogen atmosphere and had a temperature of 60° C.

After addition of the ligand-containing solution, the resulting mixture was kept at this temperature for 2 hours, after which it was quenched with acetic acid. To get rid of unreacted components and low-molecular weight by-products, the clear solution was dialyzed against excess water of Millipore quality for seven days with repeated exchange of the dialysis water. The dialysis was performed using a Spectra/Por® membrane tubing with a molecular weight cut-off of 6-8,000. After the dialysis the polymer was recovered by freeze-drying.

The nitrogen content of the new product was determined by Kjelidahl titration to be 0.24% w/w, based on the total weight of the resulting polymer. This corresponds to a MS (ligand) of 0.047.

### Example 3

Imidazole, 4.12 g (99% Aldrich, 60 mmol), was dissolved in 48 g deionized water to which 2.4 g sodium hydroxide (60 mmol) was added. The solution was subsequently heated to 50° C. and during a period of 30 minutes 5.34 ml (34 mmole) epichlorohydrin (ECH) was added dropwise from a dosimate, so that the temperature remained between 50-54° C. Following the addition of ECH, the temperature was increased to 60° C. and kept there for 30 minutes before the mixture was cooled to room temperature. Chloride analysis by titration showed a quantitative conversion of ECH.

The whole solution comprising the ligand was transferred to a dropping funnel and over a 2.5-hour period was added to an alkalized 2.5%/w/w ethyl hydroxyethyl cellulose (EHEC) solution (0.2 g NaOH and 16.7 g EHEC (MS(EO) is 2.1; DS(ethyl) is 0.8; Mw is 200,000) in 1,455.5 g of deionized water, which EHEC solution was kept under a nitrogen atmosphere and had a temperature of 60° C.

After addition of the ligand-containing solution, the resulting mixture was kept at this temperature for 2 hours, after which it was quenched with acetic acid. To get rid of unreacted components and low-molecular weight by-products, the clear solution was dialyzed against excess water of Millipore® quality for seven days with repeated exchange of the dialysis water. The dialysis was performed using a Spectra/Por® membrane tubing with a molecular weight cut off of 6-8,000. After the dialysis the polymer was recovered by freeze-drying.

The nitrogen content of the new product was determined by Kjelldahl titration to be 0.06950/w/w, based on the total weight of the resulting polymer. This corresponds to a MS (ligand) of 0.01.

### Example 4

To a stirred mixture of 170 g of dimethyl sulfoxide (DMSO), 1 g of tetrabutyl ammonium fluoride trihydrate, and 0.74 g of potassium hydroxide a 8.34 g ethyl hydroxyethyl cellulose (EHEC) solution (MS(EO) is 2.1; DS(ethyl) is 0.8; Mw is 200,000) was added over a period of 15 minutes. The mixture was heated to 60° C. and left to react for 30 minutes at this temperature. A solution of 4′-chloro-2,2′-6′,2″-terpyridine (99% Aldrich 0.24 g=0.9 mmol) in 8 g DMSO was added to this mixture and left to react under a nitrogen atmosphere for a 30-hour period, after which it was quenched with acetic acid. The reaction mixture was then poured into a mixture of 250 ml 2-propanol in 1600 ml acetone. The resulting polymer was filtered off and dried over filter paper. The nitrogen content of the new product was determined by Kjelldahl titration to be 0.115% w/w, based on the total weight of the resulting polymer. This corresponds to a MS (ligand) of 0.0758.

### Example 5

To a stirred mixture of 170 g of dimethyl sulfoxide (DMSO), 1 g of tetrabutyl ammonium fluoride trihydrate, and 0.74 g of potassium hydroxide a 8.34 g ethyl hydroxyethyl cellulose (EHEC) solution (MS(EO) is 2.1; DS(ethyl) is 0.8; Mw is 200,000) was added over a period of 15 minutes. The mixture was heated to 60° C. and left to react for 30 minutes at this temperature. A solution of 4-bromo-1H-imidazole (97% Aldrich 0.135 g=0.9 mmol) in 2 ml DMSO was added to this mixture and left to react under a nitrogen atmosphere for a 48-hour period, after which it was quenched with acetic acid. The reaction mixture was then poured into 600 ml of acetone. The resulting polymer was filtered off and dried under vacuum at 60° C. for 4 hours. The nitrogen content of the new product was determined by Kjelidahl titration to be 0.108% w/w, based on the total weight of the resulting polymer.

### Example 6

Aqueous solutions containing the polymer described in Example 3 and different concentrations of copper ions were made for viscosity measurements. The polymer and copper sulphate pentahydrate (CuSO₄. 5H₂0) were added to test tubes, which were sealed with Teflon screw caps. In all solutions the polymer concentration was 1.5% w/w, which for this polymer means a concentration of imidazole groups (c_{imidazole}) of 0.37 mM. The added concentration of CuSO₄.5H₂0 in mM and the ratio of copper ion concentration (C_{Cu}²⁺) to concentration of imidazole groups in each solution are presented in Table 1. Deionized water was used in all solutions and the pH was adjusted to 9-10 by the addition of KOH solution (conc. 5%). The viscosity was measured with a controlled stress rheometer equipped with a 4 cm 1° cone-and-plate system. The temperature during the viscosity measurement was 20±0.1° C. In all cases the viscosity was determined at the Newtonian plateau, where it is independent of the shear rate. The viscosity results are presented in Table 1.

TABLE 1

| C_{Cu}²⁺ | | Viscosity |
|---|---|---|
| (mM) | (C_{Cu}²⁺)/(C_{Imidazole}) | (mPa s) |
| 6.0 | 16 | 170 |
| 3.7 | 10 | 186 |
| 1.1 | 3 | 196 |
| 0.37 | 1 | 183 |
| 0.19 | 0.5 | 168 |
| 0.08 | 0.2 | 169 |
| 0.05 | 0.1 | 167 |
| 0 | 0 | 160 |

1. A water-soluble cellulose derivative comprising a ligand having at least two carboxylic acid groups or a ligand having at least one 5- or 6-membered ring and having at least two nitrogen atoms.

2. The cellulose derivative according to claim 1 wherein the ligand is either of the formulae (I) andor (II): 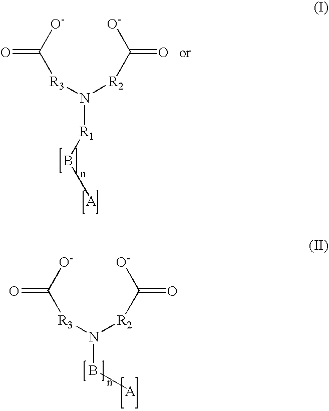
wherein R₁ is a hydrocarbon having 1 to 8 carbon atoms, optionally comprising at least one hydroxyl group, R₂ and R₃ may be the same or different and are selected from a hydrocarbon having 1 to 8 carbon atoms, [B] is attached to the cellulose backbone of the cellulose derivative [A] and selected from O, OC(O), C(O)O, C(O)—NH, NHC(O), NH—C(O)—CH₂—O, O—C(O)—NH—R₄—NH—C(O)—O, wherein R₄ is selected from substituted or unsubstituted C₁-C₈ alkylene and C₃-C₂₄ cycloalkyl, S, OSO₃, OPO₃, NH, or NR₅, wherein R₅ is a C₂-C₆ acyl or a C₁-C₄ alkyl radical, and wherein n is 0 or 1.

3. The cellulose derivative of claim 1 wherein the ligand is selected from the group consisting of imidazole, pyrimidine, purine, phenantroline, bipyridine, terpyridine, and derivatives thereof.

4. A coating composition comprising the cellulose derivative according to any one of claims 1, further comprising to 3 and a salt of a polyvalent metal ion selected from the group consisting of transition metals, Mg²⁺, Ca²⁺, and Al³⁺.

5. A process for preparing the cellulose derivatives according to of claim 1 or 2 comprising the steps of:
(a) reacting an epoxide according to either of the formulae: 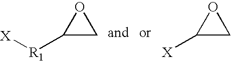
with a secondary amine of the formula: 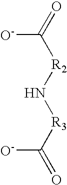
to form a ligand according to either of the formulae: 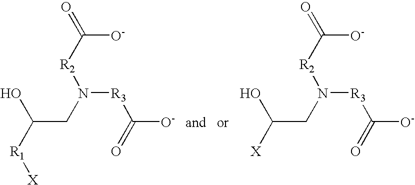
wherein R₁ is a hydrocarbon having 1 to 6 carbon atoms, optionally comprising at least one hydroxyl group, R₂ and R₃ may be the same or different and are selected from a hydrocarbon having 1 to 8 carbon atoms, and X is a halogen selected from Cl⁻, Br⁻, and I; and
(b) subsequently reacting the ligand with a cellulose derivative according to the formula: 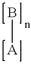
to form 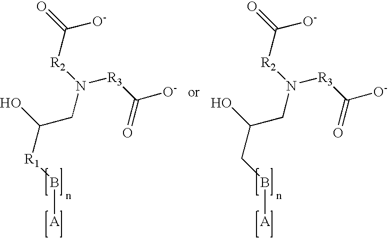
wherein R₁, R₂ and R₄ are as defined above, and [B] is attached to the cellulose backbone of the cellulose derivative [A] and selected from O, OC(O), C(O)O, C(O)—NH, NHC(O), NH—C(O)—CH₂—O, O—C(O)—NH—R₄—NH—C(O)—O, wherein R₄ is an C₁-C₈ alkylene, S, OSO₃, OPO₃, NH, or NR₅, wherein R₅ is a C₂-C₆ acyl or a C₁-C₄ alkyl radical, and wherein n is 0 or 1.
